(19) United States
(12) Patent Application Publication
(10) Pub. No.: US 2017/0182026 A1
(43) Pub. Date: Jun. 29, 2017

(54) Anti-Metastasis Treatment of Melanoma

(71) Applicant: University Hospitals Cleveland Medical Center, Cleveland, OH (US)

(72) Inventor: Joshua Arbesman, Cleveland, OH (US)

(21) Appl. No.: 15/391,516

(22) Filed: Dec. 27, 2016

Related U.S. Application Data
(60) Provisional application No. 62/272,542, filed on Dec. 29, 2015.

(51) Int. Cl. A61K 31/4706; A61K 9/00; A61K 31/045

(52) U.S. Cl. CPC A61K 31/4706; A61K 31/045; A61K 9/0053

(57) ABSTRACT

The present invention provides medicaments and methods for the treatment of melanoma. Disclosed herein are methods and uses for preventing melanoma, reducing progression of melanoma to a metastatic state, and inducing cell cycle arrest and/or apoptosis in a melanoma cell through oral, enteral, or topical administration of autophagy modulator and a gap junction intercellular communication modulator to subjects indicated to be at risk due to factor(s) such as medical history of melanoma, excessive UV exposure, or those with stage II and III melanoma.

### CROSS-REFERENCE TO RELATED APPLICATIONS

The present application claims the benefit of U.S. Provisional Patent Application No. 62/272,542, filed on Dec. 29, 2015, which is incorporated herein by reference.

### FIELD OF THE INVENTION

The present invention provides medicaments and methods for the treatment of melanoma. Disclosed herein are methods and uses for preventing melanoma, reducing progression of melanoma to a metastatic state, and inducing cell cycle arrest and/or apoptosis in a melanoma cell through oral, enteral, or topical administration of autophagy modulator and a gap junction intercellular communication modulator to subjects indicated to be at risk due to factor(s) such as medical history of melanoma, excessive UV exposure, or those with stage II and III melanoma.

### BACKGROUND OF THE INVENTION

Incidence of melanoma cases has increased 4-12 fold since the 1940s. Melanoma is now the sixth most common cancer in men, and the seventh most common cancer in women. Its incidence is increasing in all parts of the world (Parker, S et al, 1997 [1]). The 5-year survival rate for melanoma is 91.5%, which decreases depending on the stage. Spread of the disease to distant organs such as liver, bone and brain, reduces the 5-year survival to less than 12%. Standard chemotherapy regimens do not impart a significant long-term survival benefit in these patients, and chemotherapy may be associated with a degree of morbidity due to toxicity. There is an obvious need to develop new, targeted therapies for melanoma, both to treat metastatic melanoma patients, but also to prevent cancer progression in patients who do not yet demonstrate distant metastases.

### SUMMARY OF THE INVENTION

The present invention provides medicaments and methods for the treatment of melanoma. Disclosed herein are methods and uses for preventing melanoma, reducing progression of melanoma to a metastatic state, and inducing cell cycle arrest and/or apoptosis in a melanoma cell through oral, enteral, or topical administration of autophagy modulator and a gap junction intercellular communication modulator to subjects indicated to be at risk due to factor(s) such as medical history of melanoma, excessive UV exposure, or those with stage II and III melanoma. In one embodiment, the present invention contemplates a combination blockade of autophagy and gap junction intercellular communication to synergistically affect prevention of recurrence and metastasis of localized melanomas. In one embodiment, a combination of hydroxychloroquine and 1-octanol would serve as an exemplary combination for this therapy. In one embodiment, 1-octanol alone serves as a treatment for melanoma. In one embodiment, oral administration is envisioned. In one embodiment, the invention relates to therapeutics for stage II and III melanoma patients.

In one embodiment, the present invention contemplates a method for treating a subject with melanoma, comprising administering a pharmaceutically effective amount of a pharmaceutical composition including but not limited to, hydroxychloroquine and 1-octanol. In one embodiment, said melanoma cancer cells include, but are not limited to, premalignant cells, malignant cells, or multidrug-resistant cells. In one embodiment, said treatment of melanoma comprises inhibition of metastasis of a melanoma cancer cell. In one embodiment, said pharmaceutically effective amount of a pharmaceutical composition comprises 5-10 micromolar hydroxychloroquine and 1 millimolar 1-octanol.

In one embodiment, the present invention contemplates a method for treating a subject with melanoma, comprising administering a pharmaceutical composition comprising a pharmaceutically effective amount of an autophagy modulator and a gap junction intercellular communication modulator. In one embodiment, said autophagy modulator is hydroxychloroquine. In one embodiment, said gap junction intercellular communication modulator is 1-octanol. In one embodiment, said pharmaceutically effective amount of a pharmaceutical composition comprises 5-10 micromolar hydroxychloroquine and 1 millimolar 1-octanol. In one embodiment, said composition is administered orally. In one embodiment, said composition is administered before melanoma surgery. In one embodiment, said composition is administered after a lesion biopsy and before the final removal (neoadjuvant) or after the excision (adjuvant) for potentially a prolonged period of time.

In one embodiment, the present invention contemplates a pharmaceutical composition including but not limited to hydroxychloroquine and 1-octanol. In one embodiment, said formulation further comprises an effective amount of pharmaceutically acceptable carrier material.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “patient” or “subject” refers to any living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses. Although described in particular as applicable to hospitals and medical practices treating human patients, the benefits of the invention apply to veterinarian practices as well; “patient” then may be a pet or other animal, as well as a human patient.

The present invention contemplates the above-described compositions in “therapeutically effective amounts” or “pharmaceutically effective amounts”, which means that amount which, when administered to a subject or patient for treating a disease, is sufficient to effect such treatment for the disease or to ameliorate one or more symptoms of a disease or condition (e.g. ameliorate pain).

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The pharmaceutical compositions of the present invention may be prepared by formulating them in dosage forms which are suitable for peroral, rectal or nonparenteral administration, the last-mentioned including intravenous injection and administration into the cerebrospinal fluid. For this purpose, common carriers and routine formulation techniques may be employed.

“API” or “active pharmaceutical ingredient” means the substance in a pharmaceutical drug that is biologically active.

“Common carriers” means those which are employed in standard pharmaceutical preparations and includes excipients, binders and disintegrators the choice of which depends on the specific dosage form used. Typical examples of the excipient are starch, lactose, sucrose, glucose, mannitol and cellulose; illustrative binders are polyvinylpyrrolidone, starch, sucrose, hydroxypropyl cellulose and gum arabic; illustrative disintegrators include starch, agar, gelatin powder, cellulose, and CMC. Any other common excipients, binders and disintegrators may also be employed.

In addition of the carriers described above, the pharmaceutical composition of the present invention preferably contains antioxidants for the purpose of stabilizing the effective ingredient. Appropriate antioxidants may be selected from among those which are commonly incorporated in pharmaceuticals and include ascorbic acid, N-acetylcysteine, acetylcysteine, L-cystein, D, L-α-tocopherol, and natural tocopherol.

The term “Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylicacids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002) [2]. Unless otherwise specifically stated, the present invention contemplates pharmaceutically acceptable salts of the considered pro-drugs.

The term “salts”, as used herein, refers to any salt or counterion that complexes with identified compounds contained herein while retaining a desired function, e.g., biological activity. The term “counterion”, as used herein, refers to the ion that accompanies an ionic species in order to maintain electric neutrality. Examples of such salts include, but are not limited to, acid addition salts formed with inorganic acids (e.g. hydrochloric acid, hydrobromic acid, sulfuric acid, phosphoric acid, nitric acid, and the like), and salts formed with organic acids such as, but not limited to, acetic acid, oxalic acid, tartaric acid, succinic acid, malic acid, fumaric acid, maleic acid, ascorbic acid, benzoic acid, tannic acid, pamoic acid, alginic acid, polyglutamic, acid, naphthalene sulfonic acid, naphthalene disulfonic acid, and polygalacturonic acid. Pharmaceutically acceptable salts also include base addition salts which may be formed when acidic protons present are capable of reacting with inorganic or organic bases. Suitable pharmaceutically-acceptable base addition salts include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of the invention. Unless otherwise specifically stated, the present invention contemplates pharmaceutically acceptable salts of the considered compositions.

In a specific embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient or vehicle with which the active compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a subject, the pharmaceutically acceptable vehicles are preferably sterile. Water can be the vehicle when the active compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

The term “common carriers” means those which are employed in standard pharmaceutical preparations and includes excipients, binders and disintegrators the choice of which depends on the specific dosage form used. Typical examples of the excipient are starch, lactose, sucrose, glucose, mannitol and cellulose; illustrative binders are polyvinylpyrrolidone, starch, sucrose, hydroxypropyl cellulose and gum arabic; illustrative disintegrators include starch, agar, gelatin powder, cellulose, and CMC. Any other common excipients, binders and disintegrators may also be employed. In addition of the carriers described above, the pharmaceutical composition of the present invention preferably contains antioxidants for the purpose of stabilizing the effective ingredient. Appropriate antioxidants may be selected from among those which are commonly incorporated in pharmaceuticals and include ascorbic acid, N-acetylcysteine, acetylcysteine, L-cystein, D, L-α-tocopherol, and natural tocopherol.

Formulations of the pharmaceutical composition of the present invention which are suitable for peroral administration may be provided in the form of tablets, capsules, powders, granules, or suspensions in non-aqueous solutions such as syrups, emulsions or drafts, each containing one or more of the active compounds in predetermined amounts.

The granule may be provided by first preparing an intimate mixture of one or more of the active ingredients with one or more of the auxiliary components shown above, then granulating the mixture, and classifying the granules by screening through a sieve.

The tablet may be prepared by compressing or otherwise forming one or more of the active ingredients, optionally with one or more auxiliary components.

The capsule may be prepared by first making a powder or granules as an intimate mixture of one or more of the active ingredients with one or more auxiliary components, then charging the mixture into an appropriate capsule on a packing machine, etc.

The pharmaceutical composition of the present invention may be formulated as a suppository (for rectal administration) with the aid of a common carrier such a cocoa butter. The pharmaceutical composition of the present invention may also be formulated in a dosage form suitable for non-parenteral administration by packaging one or more active ingredients as dry solids in a sterile nitrogen-purged container. The resulting dry formulation may be administered to patients non-parenterally after being dispersed or dissolved in a given amount of aseptic water.

The dosage forms are preferably prepared from a mixture of the active ingredients, routine auxiliary components and one or more of the antioxidants listed above. If desired, the formulations may further contain one or more auxiliary components selected from among excipients, buffers, flavoring agents, binders, surfactants, thickening agents, and lubricants.

As used herein, the term “hydroxychloroquine” or “(RS)-2-{{4-{(7-chloroquinolin-4-yl)amino}pentyl}(ethyl)amino}ethanol” refers to a compound with the structure

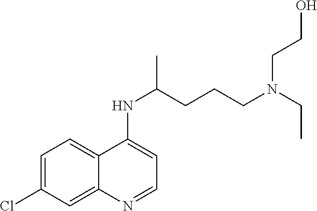 [SMILES: CCN(CCO)CCCC(C)Nc1ccnc2cc(Cl)ccc12]

or salts thereof.

As used herein, the term “1-octanol” refers to an organic compound with the molecular formula CH₃(CH₂)₇OH and the structure

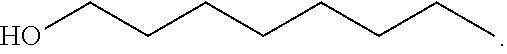

As used herein, the term “autophagy modulator” refers to an agent that modulates autophagy, an essential, conserved lysosomal degradation pathway that controls the quality of the cytoplasm by eliminating protein aggregates and damaged organelles.

As used herein, the term “gap junction intercellular communication modulator” refers to an agent that modulates gap junction intercellular communication, a process performed by gap junction channels allowing for rapid transport between cells of small molecules, such as ions and glucose. Gap junctions are formed by hemichannels on adjacent cells that are made up of connexin (Cx) proteins. A gap junction intercellular communication modulator would refer to an agent that either effects the function of gap junction intercellular communication itself or alternatively, any function of the connexin proteins, some of which have gap junction communication-independent functions.

As used herein, the term “premalignant” or “precancerous” refers to a generalized state associated with an increased risk of cancer. If left untreated, these conditions may lead to cancer. Sometimes the term “precancer” is used to describe carcinoma in situ, which are non-invasive cancers that are technically “cancers,” but have not progressed to an aggressive, invasive stage. Not all carcinoma in situ will progress to invasive disease.

As used herein, the term “malignant” refers to a tendency of a medical condition to become progressively worse, in terms of cancer. Malignancy is most familiar as a characterization of cancer. A malignant tumor contrasts with a non-cancerous benign tumor in that a malignancy is not self-limited in its growth, is capable of invading into adjacent tissues, and may be capable of spreading to distant tissues. A benign tumor has none of those properties. Malignancy in cancers is characterized by anaplasia, invasiveness, and metastasis. Malignant tumors are also characterized by genome instability, so that cancers, as assessed by whole genome sequencing, frequently have between 10,000 and 100,000 mutations in their entire genomes [3]. Cancers usually show tumour heterogeneity, containing multiple subclones [4, 5]. They also frequently have reduced expression of DNA repair enzymes due to epigenetic methylation of DNA repair genes or altered microRNAs that control DNA repair gene expression.

As used herein, the term “multidrug-resistant” refers to malignancies that do not demonstrate shrinkage or stable disease in response to multiple different drug treatments, to which they may or may not have been previously exposed.

As used herein, the term “BRAF inhibitor” or “serine/threonine-protein kinase B-Raf inhibitor” refers to a chemical or drug that inhibits the activity of wild type or mutated B-raf protein, including, but not limited to vemurafenib, dabrafenib, GDC-0879, PLX-4720, Sorafenib Tosylate, and LGX818. The B-Raf protein is involved in sending signals inside cells, which are involved in directing cell growth.

As used herein, the term “MEK inhibitor” refers to a chemical or drug that inhibits the mitogen-activated protein kinase kinase enzymes MEK1 and/or MEK2. Examples include, but are not limited to: Trametinib, Selumetinib, Binimetinib, PD-325901, and Cobimetinib.

### DESCRIPTION OF THE FIGURES

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1 depicts a schematic model demonstrating the induction of autophagosome formation when turnover is blocked vs. normal autophagic flux, and illustrating the morphological intermediates of macroautophagy. The initiation of autophagy includes the formation of a phagophore, an initial sequestering compartment, which expands into an autophagosome. Completion of an autophagosome is followed by fusion with lysosomes and degradation of its contents, allowing complete flux, or flow, through the entire pathway.

FIG. 2 shows an illustrative structural representation of both 1-octanol and hydroxychloroquine.

FIG. 3 presents exemplary data demonstrating varying expression of different connexins within eight different melanoma cell lines studied, with total AKT (t-Akt) as loading control.

FIGS. 4A&B presents exemplary data showing an assessment of connexin inhibition on autophagic flux and autophagic blockade on connexin protein levels. FIG. 4A shows A375.52 melanoma cells with treatment over 24 hours. FIG. 4B shows A375.52 cells with treatment over 24 hours. Tubulin as a loading control. H₂O=water control, CBX=Carbenoxolone, Hep=1-Eleptanol, Oct=1-octanol, D=DMSO, HCQ=Hydroxychloroquine.

FIG. 5A-C presents exemplary data showing combinatory effects of hydroxychloroquine (HCQ) and 1-octanol on melanoma cell viability and death. FIG. 5A shows the effects on cell viability as assessed by Cell-Titer Glo in two melanoma cell lines at 72 hours. 4-methyl-heptanol (1 mM) as negative control. FIG. 5B shows the effects on cell death as measured by Trypan Blue exclusion assay on A375.52 at 72 hours. FIG. 5C shows the effects on cell migration during scratch assay on A375.52 cells over 24 hours.

FIG. 6 presents exemplary data showing shRNA efficacy against autophagy proteins, Beclin1 and ATG7 in A375.52 melanoma cells with Tubulin as loading control.

FIGS. 7A&B presents exemplary data Showing cell-cell communication in Glioblastoma Stem Cells (GSCs) cells with live imaging. FIG. 7A shows a schematic depicting a microinjection strategy using an IgG to mark the parental cells (indicated by red arrow and “P”) and a reporter dye (Lucifer yellow or an fluorescent glucose analog (2NBDG)) to evaluate cell-cell communication between GSCs over time at single cell resolution. FIG. 7B shows micrographs depicting GSC to GSC (B) communication using multiple reporter dyes in cells derived from Glioblastoma specimens T4121 and T387. Communication using both reporter dyes was observed in multiple GSC specimens (data not shown).

FIG. 8 presents exemplary data showing the effect of PLX-4032 on A375.52 cells autophagic flux after treatment for 24 hours. Percent of cells demonstrating low, intermediate or high autophagic flux as determined by the ratio of mcherry:GFP in each cell.

### DESCRIPTION OF THE INVENTION

### Conventional Uses of Hydroxychloroquine and Octanol

One reference, PCT Application PCT/US2012/028567 [6], describes a combination therapy for treating cancer: a combination of an autophagy inducing agent and an autophagy inhibiting agent. The references discloses that hydroxychloroquine is an autophagy inhibiting agent. The reference further discloses that melanoma is a potential cancer to be targeted with autophagy inhibiting agents, and also describes the use of an organic solvent, including 1-octanol, in which an autophagy inhibiting agent could be dissolved to form an emulsion. The reference always describes the therapy for treating the cancer as a combination of both an autophagy inducing agent and an autophagy inhibiting agent, therefore there is no description of a composition consisting of only a pharmaceutically effective amount of an autophagy modulator and a gap junction intercellular communication modulator or consisting of hydroxychloroquine and 1-octanol.

Another reference, Lazova et al. 2012 [7], describes that autophagy presents a key target of therapeutic vulnerability in solid tumors, including melanoma, and states that hydroxychloroquine is an autophagy inhibitor. The reference does not describe either octanol or the use of a gap junction intercellular communication modulator to treat melanoma.

Another reference, ClinicalTrials Identifier: NCT00962845. describes a clinical trial using hydroxychloroquine in patients with melanoma. The hydroxychloroquine was used in combination with direct tumor surgery. The primary goal of the study was to characterize the effects of hydroxychloroquine on the modulation of markers of autophagy, as measured by p62, Beclin1, LC3, and GRp170 expression, in pre- and post-treatment tumor biopsy samples, skin samples, and peripheral blood mononuclear cell samples from patients with stage III or IV melanoma undergoing palliative or curative surgery. The reference does not describe either octanol or the use of a gap junction intercellular communication modulator to treat melanoma.

Another reference, Rangwala et al. 2014 [8], describes a trial treating melanoma with hydroxychloroquine with dose-intense temozolomide. The reference directly describes hydroxychloroquine as an autophagy inhibitor. The reference does not describe either octanol or the use of a gap junction intercellular communication modulator to treat melanoma.

Another reference, Boltz 2014 [9], describes the use of hydroxychloroquine as a chemotherapy agent against melanoma. The reference does not describe either octanol or the use of a gap junction intercellular communication modulator to treat melanoma.

Another reference, Vlahopoulos et al. 2014 [10], is a literature review evaluating chloroquines, including hydroxychloroquine, as a useful anticancer agent. In particular, the reference describes hydroxychloroquine as a useful accompanying agent with other chemotherapy agents. The reference does not describe either octanol or the use of a gap junction intercellular communication modulator to treat melanoma.

Another reference, Sartor 2012 [11], describes the combination of thiomaltol and hydroxychloroquine on melanoma cancer cells. Hydroxychloroquine is a known autophagy blocking agent. The reference does not describe either octanol or the use of a gap junction intercellular communication modulator to treat melanoma.

Another reference, Dookwah et al. 1992 [12], describes octanol being used as gap junction uncoupling agent. The reference does not describe hydroxychloroquine or the use of an autophagy modulator, nor does it describe treatment of melanoma.

Another reference, Zhang et al. 2003 [13], describes octanol being used as gap junction uncoupling agent. The reference does not describe hydroxychloroquine or the use of an autophagy modulator, nor does it describe treatment of melanoma.

Another reference, Salameh 2005 [14], is a literature review article describing the pharmacology of Gap junctions, including the targeting of cancer. Octanol is described as a gap junction uncoupling agent. The reference does not describe hydroxychloroquine or the use of an autophagy modulator, nor does it describe treatment of melanoma.

Another reference, Lin et al. 2010 [15], describes that inhibition of gap junction intercellular communication may be critical to improving the effectiveness of chemotherapies, including those that target melanoma cells. While not specifically describing octanol or any combination with hydroxychloroquine, the reference describes gap junction intercellular communication modulators in combination with chemotherapy agents to target cancer cells, including melanoma.

Another reference, United States Patent Application Publication Number US 2005-0020482 A1 [16], describes that modulating gap junctions can be useful in the treatment of cancer, although it does not specify melanoma or octanol. The reference does not describe hydroxychloroquine or the use of an autophagy modulator, nor does it describe treatment of melanoma.

### DETAILED DESCRIPTION OF THE INVENTION

The present invention provides medicaments and methods for the treatment of melanoma. Disclosed herein are methods and uses for preventing melanoma, reducing progression of melanoma to a metastatic state, and inducing cell cycle arrest and/or apoptosis in a melanoma cell through oral, enteral, or topical administration of autophagy modulator and a gap junction intercellular communication modulator to subjects indicated to be at risk due to factor(s) such as medical history of melanoma, excessive UV exposure, or those with stage II and III melanoma. In one embodiment, the present invention contemplates a combination blockade of autophagy and gap junction intercellular communication to synergistically affect prevention of recurrence and metastasis of localized melanomas. In one embodiment, a combination of hydroxychloroquine and 1-octanol would serve as an exemplary combination for this therapy. In one embodiment, 1-octanol alone serves as treatment of melanoma. In one embodiment, oral administration is envisioned. In one embodiment, the invention relates to therapeutics for stage II and III melanoma patients.

The vast majority of melanoma patients have early and intermediate stage disease, yet there is limited focus on the development of therapies preventing recurrence and metastases. This represents an opportunity for the cultivation of new therapeutics to bridge this gap for stage II and III melanoma patients. It has been estimated that approximately 69% of metastatic melanoma patients do not present with metastases, but rather started with less advanced disease [17]. Therefore, while focus on treatment of metastatic disease in melanoma is important, equal attention focusing on strategies to improve survival rates of localized disease in those that are at high risk for recurrence and metastasis is critical.

Both autophagy and gap junction intercellular communication (GJIC) have been found to be important in melanoma, with effects on melanoma growth and/or metastasis [7, 15, 18, 19]. However, while focus on these likely is geared towards metastatic melanoma, significant work has not been done on prevention of recurrences nor have combination therapies yet been established. Additionally, recent data has shown each process affecting the other, making combined therapies likely synergistic [20-22]. In one embodiment, the present invention contemplates a novel combination blockade of autophagy and gap junction intercellular communication that results in synergistic effects and provides an effective strategy for patients with localized melanomas to prevent recurrence and metastasis. Clinically available safe medications include but are not limited to hydroxychloroquine and 1-octanol. Data presented herein indicates an efficacy of a combination therapy with these hydroxychloroquine and 1-octanol on melanoma cell viability and migration, as well as cross-regulation of their respective mechanisms.

Although it is not necessary to understand the mechanism of an invention, it is believed that combined modulation of autophagy and gap junction intercellular communication (GJIC) significantly reduces melanoma cellular survival, invasion and migration by increasing reactive oxygen species (ROS) production. Although it is not necessary to understand the mechanism of an invention, it is believed that the effect of autophagy inhibition on gap junction intercellular communication protein localization and function within melanoma cell lines may be confirmed. Although it is not necessary to understand the mechanism of an invention, it is believed that GJIC inhibition on autophagic flux within melanoma cell lines may be determined. Although it is not necessary to understand the mechanism of an invention, it is believed that combined autophagy and GJIC inhibition effect on melanoma cell lines may be verified using clinically available drugs to determine the effect on melanoma cell viability, death, invasion and migration. Although it is not necessary to understand the mechanism of an invention, it is believed that the levels of ROS secondary to autophagy and GJIC inhibition may be characterized and whether blockade of ROS protects from this combination therapy determined.

Although it is not necessary to understand the mechanism of an invention, it is believed that combined autophagy and GJIC blockade in vivo reduces the recurrence and metastasis rates in melanoma transgenic mouse models. Although it is not necessary to understand the mechanism of an invention, it is believed that atransgenic inducible Tyr::CreER; BrafCa/+; Ptenlox/lox (hereafter referred to as BRAF/PTEN) mice9 that develop melanomas that have the ability to metastasize and possess a competent immune system to mimic a human system of metastasis may be used to evaluate the effect of modulation of autophagy and gap junction intercellular communication (GJIC) via combined therapy. Although it is not necessary to understand the mechanism of an invention, it is believed that tumors may be induced in such transgenic mice and the tumors subsequently surgically removed. Although it is not necessary to understand the mechanism of an invention, it is believed that treatment of transgenic mice with adjuvant combination therapy (autophagy and GJIC blockade), in comparison to monotherapy and control may provide an model for treatment and subequent monitoring for recurrence and metastasis.

### Melanoma: Clinical Considerations

Melanoma incidence is increasing, and melanoma is now the fifth most common cancer in men and the seventh most common cancer in women in the United States. Additionally, the survival rate of advanced stage disease is believed to be approximately 16% [23]. While new therapies, including BRAF and MEK inhibitors, alone or in combination, and immunotherapies, do offer extension of life, in general, they do not offer sustained remission in most patients [24, 25]. Therefore, development of new therapeutic modalities is critical and designing new strategies to prevent the development of metastatic disease is essential.

Successful adjuvant therapies in patients with intermediate stage disease would limit the number of patients that ever require therapy for metastatic disease. One recent paper indicated that of the patients that have metastatic disease, approximately 69% did not initially present with metastases.) A recent study in Sweden found that while Stage IA patients comprised approximately 46% of the population studied (with an excellent cure rate with surgery), Stage II patients composed 28% of the population, with 5 year survival rates being as low as 53% in those categorized as Stage IIC [26, 27]. Regional and metastatic disease comprise 9% and 4% of patients at presentation [28]. While the survival rates are poor in these more advanced stages, the vast majority of patients present with earlier stages. These percentages are in contrast to other cancers. For example, in breast cancer, regional and distant metastatic disease at presentation comprise 32% and 5% of patients, respectively [28]. These same statistics for colorectal cancer are 36% and 20% of patients at presentation [28]. Additionally, 30% of melanoma deaths have been associated with thin (≦1 mm Breslow depth) melanomas [29]. While some of these patients may have more advanced disease at presentation and this was not addressed in the study, it is likely that the majority of these patients did not present with advanced stage disease. Lastly, recurrences are relatively frequent in intermediate stage melanomas, particularly Stage II patients, with their 5-year Recurrence Free Survival rate only at 65.5% [30]. Another study demonstrated that 40% of patients who do not present initially with metastatic disease develop some form of recurrence, with worse prognosis once this occurs [31]. Therefore, some embodiments of the present invention focus on designing therapies and developing animal models aimed at answering questions including, but not limited to: How do we treat intermediate stage melanoma patients beyond surgery to prevent later recurrence and metastasis? Two particular cellular functions have been found to play a role in this regard: autophagy and gap junction intercellular communication (GJIC).

### Autophagy

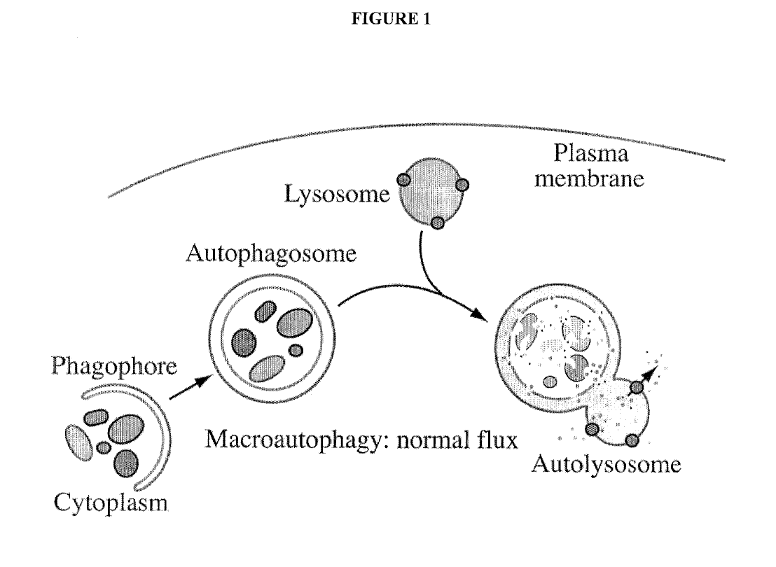

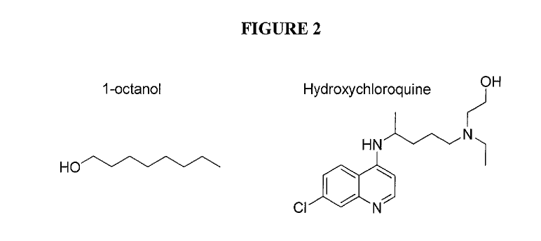

Autophagy is a process that serves to degrade defective proteins and cellular organelles [32]. It occurs through the formation of a double-membrane into autophagic vesicles/autophagosomes within various portions of the cytosol, often near damaged organelles and older proteins. These vesicles then fuse with the lysosome, with subsequent degradation of the autophagosome's contents as illustrated in FIG. 1 [33]. Upregulation of autophagy occurs in times of cell nutrient and energy demand or starvation. Cellular stresses, such as hypoxia, or a greater requirement for clearance of damaged organelles or proteins, such as in neurodegenerative diseases, can also lead to increased autophagic flux [34, 35]. Given that cancerous cells have high stress/nutrient requirements, autophagy may play a role in cancer. While some studies report that tumor development is linked to down regulation of autophagy [36-39], alternative reports show more advanced tumors have been found to have increased levels of autophagic flux [40, 41]. Recent work has investigated autophagy's role within melanoma, both in preclinical and clinical studies. Studies have had discordant results regarding LC3 expression, a marker of autophagy, in melanoma samples, with some indicating that increased expression is associated with worse prognostic indicators [7, 18], while others demonstrating the opposite result [42, 43]. However, assessment of autophagy and autophagic flux within human tissue specimens remains a challenging exercise and is not as well developed [33]. Preclinical work with in vitro and mouse models have yielded more information, with one report indicating that melanoma cells that are more aggressive phenotypically have increased levels of autophagy and addition of autophagy inhibitors resulted in increased cell death [41]. Further, autophagy inhibition resulted in a reduction in tumor growth in xenografted human melanoma cells [44]. Lastly, a recent report found that overexpression of BRAFV600E in melanocytes along with blocked Atg5 (a key autophagy protein) expression with shRNA yielded greater proliferation and likely blockage of oncogene-induced senescence compared to BRAFV600E overexpression alone [38]. While the results of this work have resulted in renewed focus on hydroxychloroquine as an anticancer agent, given its ability to inhibit autophagy, embodiments of the present invention repurpose this drug by targeting another process. Given recent evidence of melanoma escape mechanisms from single agent therapy in metastatic melanoma, as seen in resistance acquired in BRAF mutation-positive metastatic melanoma patients being treated with vemurafenib, that the presently disclosed combination therapy may improve adjuvant therapy as well.

Gap Junction Communication

Gap junctions are believed to mediate direct cell-cell comunication, allowing for a rapid transport of small molecules between cells, such as ions and glucose [45, 46]. Gap junctions may be formed by hemichannels on adjacent cells that may include connexin (Cx) proteins [46].

While connexins are believed to play a role in controlling the transport of molecules between cells, recent work has identified gap junction communication-independent roles of connexin proteins, including involvement in tumor development/tumor suppressor roles and tumor metastasis [46]. There is conflicting evidence regarding the effect of connexin expression within melanoma, somewhat dependent on the particular connexin studied. For example, it has been shown that in B16 melanoma cells expressing Cx26 that connexin blockade decreased brain metastases and the number and size of tumors associated with the vasculature [19]. Further, Lin et al. found that GJIC between melanoma cells and astrocytes protected melanoma cells from death when exposed to chemotherapeutic agents, likely indicating that GJIC helps protect melanoma cells that have metastasized to the brain [15]. However, another study found that overexpression of Cx43 reduces melanoma cell proliferation and anchorange-independent growth [47]. Lastly, recently there has been evidence demonstrating reciprocal modulation between autophagy and connexins. Macroautophagy has been found to contribute to the degradation of connexin proteins, and blockade of autophagy leads to increased levels and function of connexin proteins [20, 22]. Additionally, downregulation of Cx43 was found to lead to increased autophagic flux, while it appeared that pharmacologic blockage of GJIC with 18α glycyrrhetinic acid, a connexin inhibitor, led to decreased autophagic flux [21]. Although it is not necessary to understand the mechanism of an invention, it is believed that this reciprocal modulation between autophagy and connexins within melanoma can be exploited to develop novel combination therapies to prevent metastasis and recurrence in intermediate stage melanoma patients.

### Preliminary Studies

Based upon a previous literature of autophagy and GJIC within melanoma, and the interplay between the two processes, some embodiments of the present invention elucidates a relationship between these two processes within melanoma and an effect of a combination blockade as a novel adjuvant therapy.

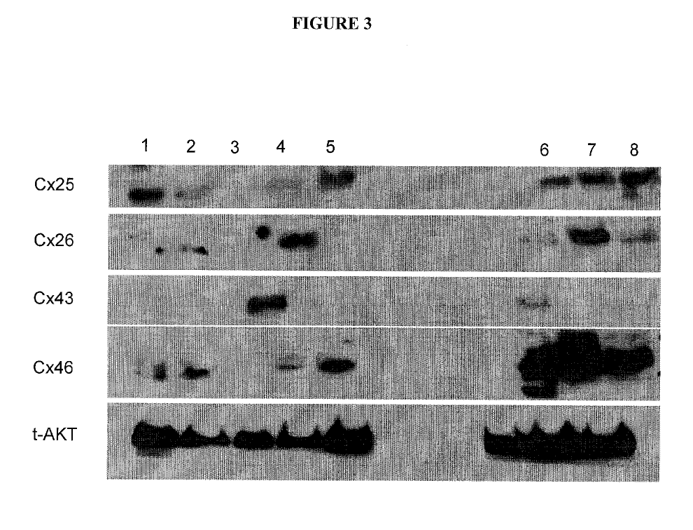

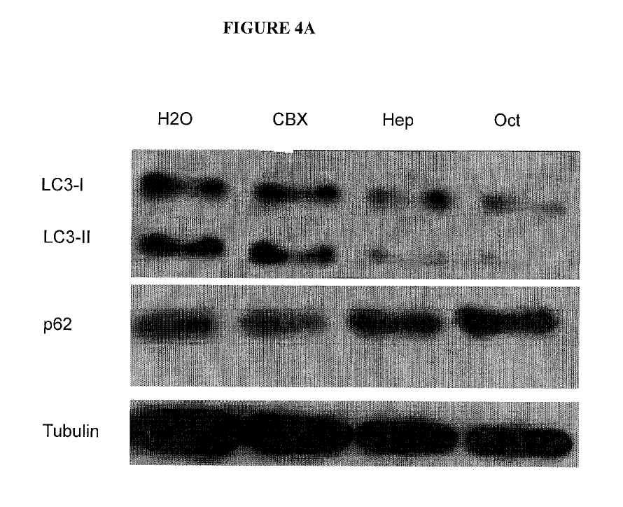

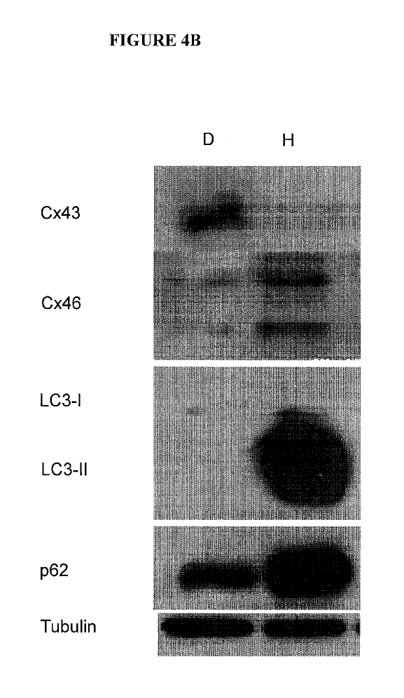

Connexin protein levels were examined within various melanoma cell lines, see FIG. 3. As can be seen, there is significant connexin expression within multiple cell lines, but it is not a uniform pattern. Connexins 43 and 26 has been studied previously in melanoma, and both also have shown relationships with autophagy [15, 19-22, 47]. Further, connexin 43 and 46 expression has been shown to be reciprocally related [48]. GJIC inhibition was explored, using three known connexin inhibitors, 1-octanol, 1-heptanol and carbenoxolone, the former of which is clinically available and is labeled as a food additive by the Food and Drug Administration, which is known to affect autophagic flux. GJIC inhibition was measured by changes noted in LC3 II/I ratios and p62 levels. (FIG. 4A). As can be seen, these compounds may affect autophagic flux. For example, carbenoxolone (CBX) decreased p62 levels somewhat, indicative of potentially increased autophagic flux, without clear changes in LC3 II/I ratio, while 1-octanol (and 1-heptanol to a lesser extent, if at all) increased p62 levels (indicative of decreased autophagic flux) with changes in total LC3 levels.

To obtain a better analysis of autophagic flux, rather than a static time point, mcherry-GFP-LC3 containing plasmids may also be used, which are further described below. These fluorescent compounds more clearly demonstrate the effect on autophagic flux, i.e. the process going to completion, and can be easily measured at multiple time points.

Connexin protein levels were also assessed for possible modification of autophagic flux. For this purpose, hydroxychloroquine, a known autophagy inhibitor and clinically available medication was used. Although it is not necessary to understand the mechanism of an invention, it is believed that hydroxychloroquine affects autophagy modulation and connexin proteins.

Specific autophagy inhibition may be assessed using shRNA (infra). The data shows that hydroxychloroquine does affect protein levels of connexins, but not consistently, where some connexin protein levels decreased while other connexin protein levels increased. FIG. 4B for example, the opposite effects on levels of connexins 43 and 46 is not surprising, as in another system these particular connexins were shown to have a reciprocal relationship [48].

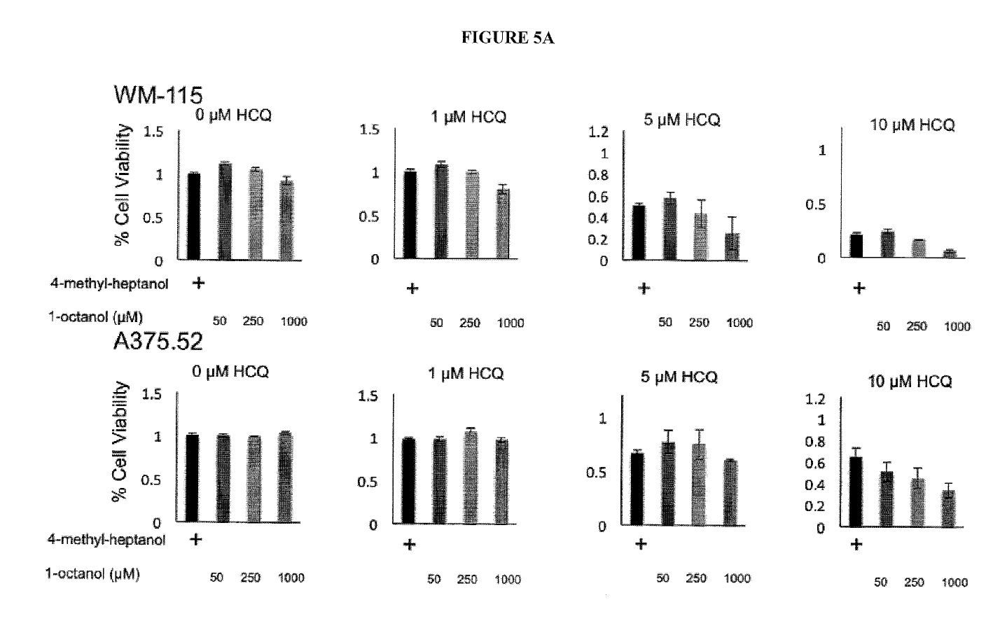

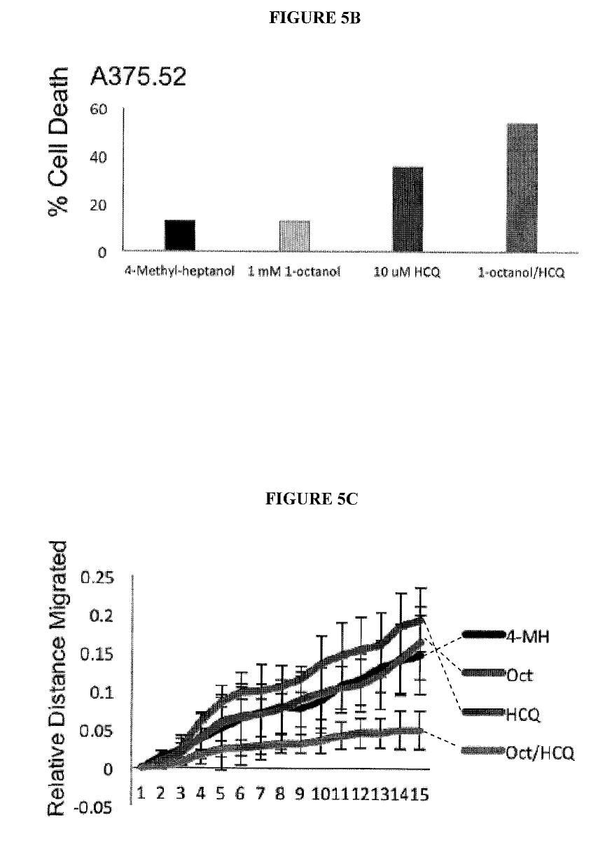

The effect of a combined inhibition of GJIC and autophagy was assessed, making use of Promega Cell-Titer Glo and Trypan blue exclusion assay to assess cell viability and cell death, respectively (FIG. 5A and FIG. 5B). While only small effects on viability and death are observed with 1-octanol, there is a synergistic effect when hydroxychloroquine and 1-octanol are combined. Additionally, cell migration was measured using a scratch assay and found synergistic effects when hydroxychloroquine and 1-octanal are combined, while no effect was observed when either agent was administered alone. (FIG. 5C) Cell migration was measured as distance traveled from the edge of a wound and not as percent wound closure.

### Pharmaceutical Compositions

The present invention further provides pharmaceutical compositions (e.g., comprising the compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### EXPERIMENTAL

### Example 1

Relationship Between Autophagy and Gap Junction Intercellular Communication in Melanoma Using shRNA

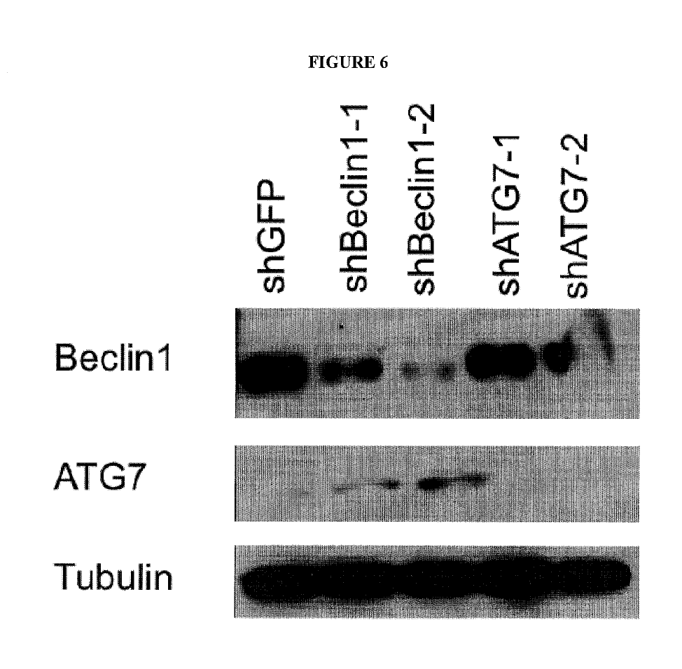

Preliminary data identifies that autophagy blockade using a pharmacologic and clinically available inhibitor, hydroxychloroquine, affects the protein levels of specific connexins. However, these results were discordant. It is proposed that: 1) Primers for PCR for nineteen individual connexin proteins have been validated. To determine the effect on the expression level of these proteins, pharmacologic blockade, hydroxychloroquine, and genetic blockade, using shRNA, of autophagy will be employedshRNA has been used successfully against Atg7 and Beclin 1 (two separate shRNAs each), two key autophagy proteins, see FIG. 6. The autophagic blockade may primarily have an effect on protein levels due to its role in connexin degradation, and therefore, analysis of other connexin proteins will be expanded using western blot as well. This too would be performed with pharmacologic and genetic blockade.

### Example 2

Evaluating the Functional Status of Overall Connexin Activity after Autophagy Inhibition

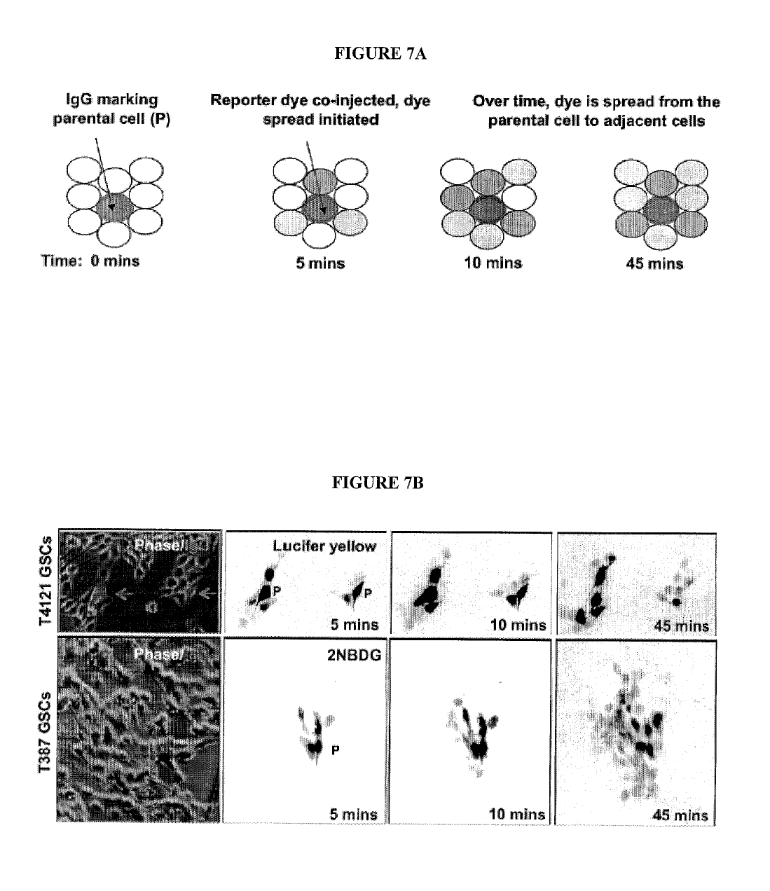

The functional status of overall connexin activity after autophagy inhibition will be addressed. This is important, as seen in the preliminary data, the effect of autophagy inhibition by hydroxychloroquine has different effects on different connexin protein levels. To measure this, a microinjection imaging assay will be used. One cell is co-injected with a large fluorescent IgG that is unable to leave the parental cell along with a reporter dye that is capable of spreading to adjacent cells. Combining this with time-lapse microscopy, spread of the reporter dye (Lucifer yellow, 2NBDG-a fluorescent glucose analog) can be monitored, see FIGS. 7A&B. The spread of the dye is attenuated when using gap junction inhibitors, including carbenoxolone and 1-octanol. Utilization of multiple assessments of the gap junction intercellular communication, i.e. mRNA expression, protein levels and functional activity levels may pinpoint the particular modulation of GJIC that occurs with autophagic blockade in melanoma cells. Alternative approaches: What happens if autophagy inhibition does not affect connexins? While preliminary data indicates that there is some effect on connexin protein expression, as described expanding these results to other connexins would be a next step, as these may be more affected by autophagic degradation. Additionally, shRNA against shATG5, another key autophagy protein, could be utilized to determine if there were different effects.

### Example 3

Evaluating the Reciprocal Relationship that Connexin Modulation has on Autophagic Flux

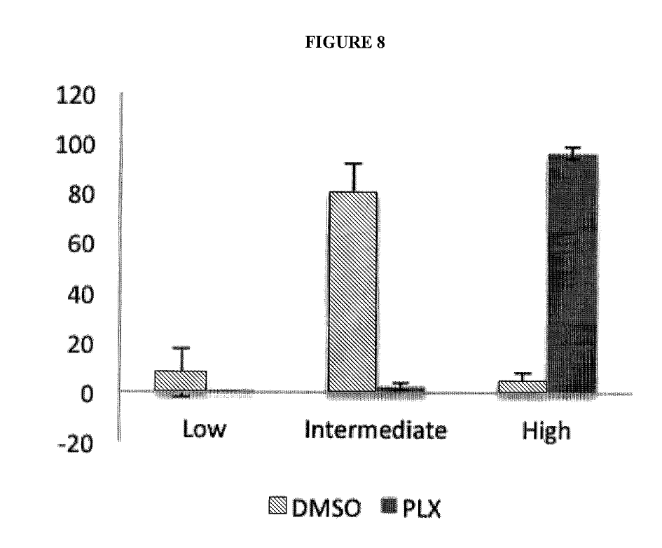

The reciprocal relationship that connexin modulation has on autophagic flux is important to evaluate. As previously shown in FIG. 4A, use of different connexin inhibitors yielded differing effects on autophagic flux. Evaluation of other connexin inhibitors, including mefloquine (a clinically available malaria medication) may be the next step. The differing specificity of different inhibitors to particular connexins (while some are pan-inhibitors) may explain the differing effects on autophagic flux. For example, mefloquine has been found to block activity of Cx 36 and 50, but would require much higher doses to affect connexins 26, 32, and 43 [49]. To confirm the effective blockade of connexin activity, a microinjection assay described in Example 2 will be used. Western blots of LC3 and p62 protein levels will be used to assess the effects of connexin inhibition on autophagic flux, and this will be done with and without a vacuolar-ATPase inhibitor, such as Bafilomycin A1. Additionally, stably transfected cells with mcherry-GFP-LC3 containing plasmids, which will mark autophagosomes (GFP & mcherry)/autolysosomes (mcherry alone) that can allow us to monitor the effects of connexin inhibition on autophagic flux will be employed, as previously described [33]. This reporter system utilizes the higher sensitivity of GFP to the acidic environment of lysosomes compared to mCherry. Therefore, cells with greater levels of completed autophagic flux have greater mcherry:GFP ratios, secondary to autophagosome fusion with lysosomes. This has been used successfully in investigating the effects of vemurafenib (PLX-4032) on autophagic flux and determine levels of cells with low and high autophagic flux using flow cytometry, see FIG. 8.

### Example 4

Genetic Blockade of Connexins Using shRNA Against Connexins 26, 43 and 46

A genetic blockade of connexins using shRNA against connexins 26, 43 and 46 in melanoma cells will be used to determine if this mimics pharmacologic blockade or not with regards to autophagic flux, with the first two connexins noted already having been deemed important in melanoma and the latter as it is reciprocally regulated with connexin 43.

### Example 5

Genetic Blockade of Connexins Using shRNA Against Connexins 26, 43 and 46

As described in the preliminary data, successful blockade of autophagy and GJIC is thought to be combinatory and results in significant decreases in melanoma cell viability and increases in cell death. To determine whether the change in cell viability is dependent on increased cell death and/or decreased cell proliferation, the following will be measured: 1) Effects on cell death using Trypan blue exclusion assay and with Caspase 3/7 Activity (to determine apoptosis) and Annexin V assays. 2) Cell proliferation using a cell growth assay with crystal violet. 3) Cell viability at extended time points. These measurements will be important especially in determining adjuvant therapy, as it often occurs over an extended period of time, and if continued cell death occurs over time, this would help inform potentially the efficacy of continued therapy in patients. These assays will be done using multiple connexin inhibitors, focusing on agents that are clinically available, such as 1-octanol and mefloquine. The combination blockade of both processes will be evaluated for effectiveness in BRAF mutant and BRAF wild type cell lines, making the treatment useful in multiple tumor types. Finally, double transfection will be performed using shRNA affecting GJIC (against Cx26, 43 & 46) and autophagy (against Atg7 and Beclin1), helping isolate the particular connexins that modulate this interaction and synergy. Cell viability will then be assessed.

### Example 6

Effect on Melanoma Cell Proliferation and Migration

Further, melanoma metastasis/recurrence is not solely dependent on cell survival, but is also affected by cellular ability to migrate and invade other tissue. Therefore, we will build on our preliminary data indicating effect on melanoma cell proliferation and migration to determine whether this combination therapy affects melanoma cell invasion and confirm effects on migration, important markers of metastasis. Matrigel invasion chambers, and single cell migration and scratch assays will be used, which have been used extensively in the art [50]. Additionally, the combination therapy will be tested in 3-D culture to see if there is a difference, as autophagy inhibition has been shown to be more effective in 3-D culture [41].

### Example 7

Assessing the Levels of Reactive Oxygen Species in Treated Cells

GJIC and autophagy have been shown to be critical to cellular response to reactive oxygen species [51, 52]. Increased ROS is well known to induce cell death and apoptosis [53]. However, given the reciprocal modulation by the two processes, it is possible that blockade of one process will not be as damaging to the cell through increased ROS, as the other compensatory process will be potentially increased. Yet, combined blockade may yield inhibition of two ROS response mechanisms and may likely yield a significant effect. Therefore, evaluation of the mechanism of decreased melanoma cell viability on the increased level of ROS is an important focus. Assessing the levels of reactive oxygen species in cells treated with single therapies targeting autophagy and GJIC and combination therapy will be accomplished using Life Technologies CellROX® Green Reagent in combination with flow cytometry. Additionally, use of ROS scavengers, such as N-acetyl-cysteine, may be used to determine whether this will reverse the effect on cell death.

### Example 8

Assessing the Senescence

Another process that may be affected by both autophagy and connexins is senescence [51, 54]. Therefore, as inhibition of both processes induces senescence, it could be that combined blockade creates a significant enough pressure towards senescence. It is possible that this could be mediated by either a ROS-dependent mechanism or a ROS-independent mechanism. Levels of senescence may be assessed by a variety of measures, including but not limited to senescence-associated-β-galactosidase staining and quantification, senescence-associated heterochromatic foci and examination of cell morphology, after combination treatment.

### Example 9

Therapy Evaluation in Melanoma Mouse Models

To determine the in vivo effect of combined autophagy and gap junction intracellular communication blockade on the prevention of recurrence and metastasis, melanoma mouse models will be employed. A transgenic mouse melanoma model harboring a constitutively active BRAF V600E oncogene (Tyr::CreER; Braf CA/+; Ptenlox/lox) and PTEN loss of function, and importantly, develops lesions that are biologically similar to human disease [55] will be used. Melanoma development may be initiated by a single cutaneous application of 4-hydroxytamoxifen (4-HT) and primary and metastatic tumors develop within 25-50 days after stimulation. 100% of mice develop metastases to the regional draining lymph nodes [55]. The development of melanomas per mouse will be induced, and once the melanoma develops, growth rates will be recorded. Tumors will be excised when they reach comparable sizes with the mice subjected to survival surgery to remove the primary tumors. The mice will then be randomized into four groups: treatment with vehicle, hydroxychloroquine 60 mg/kg/day alone intraperitoneally, 1-octanol 350 mg/kg 3×/week intraperitoneally alone and combination therapy with hydroxychloroquine and 1-octanol, as previously described [44, 56]. The mice will then be monitored for recurrence and metastasis twice weekly. The latter will be monitored for by noting signs of morbidity from the metastasis, including change in posture, difficulty breathing or decreased feeding. At this time, the mice will be sacrificed and major organs and lymph nodes will be examined for the presence and number of metastases. Additionally, a control group will also be sacrificed at the time of surgical removal of the tumors to assess for any metastases present. Tissue will be stained with S100 as previously described, to assess for metastases [57]. If there are micro-metastases, this experiment will help determine whether this therapy will prevent growth and further metastases of what is present. If there are no micro-metastases present, an assessment of whether single cells that may not be able to be detected microscopically will be prevented from growing and metastasizing should still be possible. This will mimic the two clinical scenarios in patients, where patients either have micrometastases at the time of surgical removal (but no distant metastases-Stage III) or do not have any evidence of metastases at time of surgical removal of melanoma, but do still recur and/or metastasize later (Stage II). The tumors will also be stained for markers of autophagy, including LC3 and p62 and for the presence of connexins, including, but not limited to, connexins 26, 43 and 46. Statistical analysis: Kaplan-Meier survival curves summarizing time to metastasis will be calculated for each treatment group with the logrank test used to make between-group comparisons. With 20 animals per group power will be 80% to detect a six-week increase in median survival time, assuming 41% or more animals in the treated groups are metastasis-free at the time. While attempts will be made surgically remove all of the tumors in each mouse, there is sometimes additional development of distant tumors on the mice, as the can be spread by grooming (inducing another tumor elsewhere). If this cannot be controlled, one tumor from a mouse will be excised and transplanted to another non-transgenic mouse of the same background. This will be allowed to grow and subsequently surgically excised. The same treatment protocol as described above will be then carried out.

### Example 10

Therapy Evaluation in Human Melanoma Samples

An examination of markers of autophagy and GJIC in human melanoma samples may help to determine the relationship within tumors of these two processes. Additionally, if combined inhibition of autophagy and GJIC were successful in preventing metastasis, an attempt to adapt the clinically available inhibitors to human use.

Thus, specific systems, devices and methods of an anti-metastasis treatment of melanoma have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### REFERENCES

1. Parker, S. L. et al. (1997) “Cancer Statistics, 1997,” CA. Cancer J. Clin. 47(1), 5-27.
2. Stahl, P. H. and Wermuth, C. G., (Eds.) (2002) Handbook of Pharmaceutical Salts: Properties Selection and Use, Verlag Helvetica Chimica Acta/Wiley-VCH, Zurich.
3. Tuna, M. and Amos, C. I. (2013) “Genomic Sequencing in Cancer,” Cancer Lett. 340(2), 10.1016/j.canlet.2012.1011.1004.
4. Swanton, C. (2012) “Intratumour Heterogeneity: Evolution through Space and Time,” Cancer Res. 72(19), 4875-4882.
5. Sabaawy, H. E. (2013) “Genetic Heterogeneity and Clonal Evolution of Tumor Cells and Their Impact on Precision Cancer Medicine,” J. Leuk. 1(4), 1000124.
6. Stern, S. T. et al. “Combination Chemotherapy for Treating Cancer,” WIPO PCT Patent Publication Number WO/2012/125486, Application PCT/US2012/028567, filed Mar. 9, 2012. (published Sep. 20, 2012).
7. Lazova, R. et al. (2012) “Punctate LC3B Expression Is a Common Feature of Solid Tumors and Associated with Proliferation, Metastasis, and Poor Outcome,” Clin. Cancer Res. 18(2), 370-379.
8. Rangwala, R. et al. (2014) “Phase I Trial of Hydroxychloroquine with Dose-Intense Temozolomide in Patients with Advanced Solid Tumors and Melanoma,” Autophagy 10(8), 1369-1379.
9. Boltz, K. (2014) “Malaria Drug May Help Overcome Resistance to Melanoma BRAF Drugs,” Oncology Nurse Advisor.
10. Vlahopoulos, S. et al. (2014) “New Use for Old Drugs? Prospective Targets of Chloroquines in Cancer Therapy,” Curr Drug Targets 15(9), 843-851.
11. Sartor, Z. R. (2012) “Synergistic Effects of Hydroxychloroquine on the Activity of Thiomaltol against Melanoma Cancer Cells,” p 22, Baylor University, Waco, Tex.
12. Dookwah, H. D. et al. (1992) “Gap Junctions in Myometrial Cell Cultures: Evidence for Modulation by Cyclic Adenosine 3′:5′-Monophosphate,” Biol. Reprod. 47(3), 397-407.
13. Zhang, W. et al. (2003) “Intercellular Calcium Waves in Cultured Enteric Glia from Neonatal Guinea Pig,” Glia 42(3), 252-262.
14. Salameh, A. and Dhein, S. (2005) “Pharmacology of Gap Junctions. New Pharmacological Targets for Treatment of Arrhythmia, Seizure and Cancer?,” Biochim. Biophys. Acta Biomembr. 1719(1-2), 36-58.
15. Lin, Q. et al. (2010) “Reactive Astrocytes Protect Melanoma Cells from Chemotherapy by Sequestering Intracellular Calcium through Gap Junction Communication Channels,” Neoplasia 12(9), 748-754.
16. Phipps, J. et al. “Methods for Modulating Gap Junctions,” United States Patent Application Publication Number US 2005-0020482 A1, application Ser. No. 10/480,685, filed Aug. 25, 2004. (published Jan. 27, 2005).
17. Tas, F. (2012) “Metastatic Behavior in Melanoma: Timing, Pattern, Survival, and Influencing Factors,” J. Oncol. 2012, 647684.
18. Han, C. et al. (2011) “Overexpression of Microtubule-Associated Protein-1 Light Chain 3 Is Associated with Melanoma Metastasis and Vasculogenic Mimicry,” Tohoku J. Exp. Med. 223(4), 243-251.
19. Stoletov, K. et al. (2013) “Role of Connexins in Metastatic Breast Cancer and Melanoma Brain Colonization,” J. Cell Sci. 126(4), 904-913.
20. Bejarano, E. et al. (2012) “Autophagy Modulates Dynamics of Connexins at the Plasma Membrane in a Ubiquitin-Dependent Manner,” Mol. Biol. Cell 23(11), 2156-2169.
21. Bejarano, E. et al. (2014) “Connexins Modulate Autophagosome Biogenesis,” Nat. Cell Biol. 16(5), 401-414.
22. Fong, J. T. et al. (2012) “Internalized Gap Junctions Are Degraded by Autophagy,” Autophagy 8(5), 794-811.
23. Cancer Atlanta: American Cancer Society (2014) Facts & Figures 2014.
24. Flaherty, K. T. et al. (2012) “Combined BRAF and Mek Inhibition in Melanoma with BRAF V600 Mutations,” N Engl. J Med. 367(18), 1694-1703.
25. Robert, C. et al. (2011) “Ipilimumab Plus Dacarbazine for Previously Untreated Metastatic Melanoma,” N Engl. J. Med. 364(26), 2517-2526.
26. Andersson, T. M. L. et al. (2014) “Estimating the Cure Proportion of Malignant Melanoma, an Alternative Approach to Assess Long Term Survival: A Population-Based Study,” Cancer Epidemiol. 38(1), 93-99.
27. Balch, C. M. et al. (2009) “Final Version of 2009 Ajcc Melanoma Staging and Classification,” J. Clin. Oncol. 27(36), 6199-6206.
28. Howlader, N. N. et al. (2014) “Seer Cancer Statistics Review, 1975-2011,” April 2014 ed., National Cancer Institute.
29. Jemal, A. et al. (2011) “Recent Trends in Cutaneous Melanoma Incidence and Death Rates in the United States, 1992-2006,” J. Am. Acad. Dermatol. 65(5, Supplement 1), S17.ell-S17.ell.
30. Leiter, U. et al. (2012) “Hazard Rates for Recurrent and Secondary Cutaneous Melanoma: An Analysis of 33,384 Patients in the German Central Malignant Melanoma Registry,” J Am. Acad. Dermatol. 66(1), 37-45.
31. Salama, A. K. S. et al. (2013) “Hazard-Rate Analysis and Patterns of Recurrence in Early Stage Melanoma: Moving Towards a Rationally Designed Surveillance Strategy,” PLoS. ONE 8(3), e57665.
32. Yang, Z. J. et al. (2011) “Autophagy Modulation for Cancer Therapy,” Cancer Biol. Ther. 11(2), 169-176.
33. Klionsky, D. J. et al. (2012) “Guidelines for the Use and Interpretation of Assays for Monitoring Autophagy,” Autophagy 8(4), 445-544.
34. Amaravadi, R. K. et al. (2011) “Principles and Current Strategies for Targeting Autophagy for Cancer Treatment,” Clin. Cancer Res. 17(4), 654-666.
35. Sridhar, S. et al. (2012) “Autophagy and Disease: Always Two Sides to a Problem,” J Pathol. 226(2), 255-273.
36. Liang, C. et al. (2006) “Autophagic and Tumour Suppressor Activity of a Novel Beclin1-Binding Protein Uvrag,” Nat. Cell Biol. 8(7), 688-698.
37. Qu, X. et al. (2003) “Promotion of Tumorigenesis by Heterozygous Disruption of the Beclin 1 Autophagy Gene,” J. Clin. Invest. 112(12), 1809-1820.
38. Liu, H. et al. (2013) “Down-Regulation of Autophagy-Related Protein 5 (Atg5) Contributes to the Pathogenesis of Early-Stage Cutaneous Melanoma,” Sci. Transl. Med. 5(202), 202ra123-202ra123.
39. Takamura, A. et al. (2011) “Autophagy-Deficient Mice Develop Multiple Liver Tumors,” Genes Dev 25(8), 795-800.
40. Guo, J. Y. et al. (2011) “Activated Ras Requires Autophagy to Maintain Oxidative Metabolism and Tumorigenesis,” Genes Dev 25(5), 460-470.
41. Ma, X.-H. et al. (2011) “Measurements of Tumor Cell Autophagy Predict Invasiveness, Resistance to Chemotherapy, and Survival in Melanoma,” Clin. Cancer Res. 17(10), 3478-3489.
42. Miracco, C. et al. (2010) “Beclin 1 and Lc3 Autophagic Gene Expression in Cutaneous Melanocytic Lesions,” Hum. Pathol. 41(4), 503-512.
43. Sivridis, E. et al. (2011) “Beclin-1 and Lc3a Expression in Cutaneous Malignant Melanomas: A Biphasic Survival Pattern for Beclin-1,” Melanoma Res. 21(3), 188-195.
44. McAfee, Q. et al. (2012) “Autophagy Inhibitor Lys05 Has Single-Agent Antitumor Activity and Reproduces the Phenotype of a Genetic Autophagy Deficiency,” Proc. Natl. Acad. Sci. U.S.A 109(21), 8253-8258.
45. Andang, M. and Lendahl, U. (2008) “Ion Fluxes and Neurotransmitters Signaling in Neural Development,” Curr Opin. Neurobiol. 18(3), 232-236.
46. Dbouk, H. A. et al. (2009) “Connexins: A Myriad of Functions Extending Beyond Assembly of Gap Junction Channels,” Cell Commun. Signal. 7, 4-4.
47. Ableser, M. J. et al. (2014) “Connexin43 Reduces Melanoma Growth within a Keratinocyte Microenvironment and During Tumorigenesis in Vivo,” J. Biol. Chem. 289(3), 1592-1603.
48. Banerjee, D. et al. (2011) “Investigation of the Reciprocal Relationship between the Expression of Two Gap Junction Connexin Proteins, Connexin46 and Connexin43,” J. Biol. Chem. 286(27), 24519-24533.
49. Cruikshank, S. J. et al. (2004) “Potent Block of Cx36 and Cx50 Gap Junction Channels by Mefloquine,” Proc. Natl. Acad. Sci. U.S.A 101(33), 12364-12369.
50. Shaverdashvili, K. et al. (2014) “Mtl-Mmp Modulates Melanoma Cell Dissemination and Metastasis through Activation of Mmp2 and Rac1,” Pigment Cell Melanoma Res. 27(2), 287-296.
51. Taniguchi Ishikawa, E. et al. (2012) “Connexin-43 Prevents Hematopoietic Stem Cell Senescence through Transfer of Reactive Oxygen Species to Bone Marrow Stromal Cells,” Proc. Natl. Acad. Sci. U S. A. 109(23), 9071-9076.
52. Kaminskyy, V. O. et al. (2012) “Suppression of Basal Autophagy Reduces Lung Cancer Cell Proliferation and Enhances Caspase-Dependent and -Independent Apoptosis by Stimulating Ros Formation,” Autophagy 8(7), 1032-1044.
53. Simon, H. U. et al. (2000) “Role of Reactive Oxygen Species (Ros) in Apoptosis Induction,” Apoptosis 5(5), 415-418.
54. Zhang, C. F. et al. (2015) “Suppression of Autophagy Dysregulates the Antioxidant Response and Causes Premature Senescence of Melanocytes,” J. Invest. Dermatol. 135(5), 1348-1357.
55. Dankort, D. et al. (2009) “Braf(V600e) Cooperates with Pten Silencing to Elicit Metastatic Melanoma,” Nat. Genet. 41(5), 544-552.
56. Martin, F. C. and Handforth, A. (2006) “Carbenoxolone and Mefloquine Suppress Tremor in the Harmaline Mouse Model of Essential Tremor,” Mov. Disord. 21(10), 1641-1649.
57. Scortegagna, M. et al. (2014) “Genetic Inactivation or Pharmacological Inhibition of Pdkl Delays Development and Inhibits Metastasis of Braf(V600e)::Pten−/− Melanoma,” Oncogene 33(34), 4330-4339.

We claim:

1. A method for treating a subject with melanoma, comprising administering a pharmaceutically effective amount of a pharmaceutical composition consisting of hydroxychloroquine, 1-octanol, binders and excipients.

2. The method of claim 1, wherein the melanoma cancer cells are selected from the group consisting of premalignant cells, malignant cells, or multidrug-resistant cells.

3. The method of claim 1, wherein treating comprises inhibition of metastasis of a melanoma cancer cell.

4. A method for treating a subject with melanoma, comprising administering a pharmaceutical composition consisting of pharmaceutically effective amount of an autophagy modulator, a gap junction intercellular communication modulator, binders and excipients.

5. The method of claim 4, wherein said autophagy modulator is hydroxychloroquine.

6. The method of claim 4, wherein said gap junction intercellular communication modulator is 1-octanol.

7. The method of claim 4, wherein said pharmaceutically effective amount of a pharmaceutical composition comprises 5-10 micromolar hydroxychloroquine and 1 millimolar 1-octanol.

8. The method of claim 4, wherein said composition is administered orally.

9. The method of claim 4, wherein said composition is administered before melanoma surgery.

10. The method of claim 4, wherein said composition is administered after a lesion biopsy and before the final removal.

11. The method of claim 4, wherein said composition is administered after the excision.

12. An anti-cancer combination for use in treating a subject having melanoma, comprising a pharmaceutical composition consisting of hydroxychloroquine and 1-octanol.

13. The formulation of claim 12, wherein said formulation further comprises an effective amount of pharmaceutically acceptable carrier material.
